(19) United States
(12) Patent Application Publication
(10) Pub. No.: US 2005/0187211 A1
(43) Pub. Date: Aug. 25, 2005

(54) N-arylsalkyl-carboxamide compositions and methods

(76) Inventor: Edward T. Wei, Berkeley, CA (US)

Correspondence Address: Edward T. Wei, 480 Grizzly Peak Blvd., Berkeley, CA, 94708, US

(21) Appl. No.: 11/064,358

(22) Filed: Feb. 22, 2005

Related U.S. Application Data
(60) Provisional application No. 60/547,263, filed on Feb. 23, 2004.

(51) Int. Cl. A61K 31/54; A61K 31/24

(52) U.S. Cl. 514/223.8; 514/534

(57) ABSTRACT

N-(Substituted-aryl-alkyl)-cycloalkyl carboxamide compositions are disclosed that target molecular elements on sensory nerves and on secretory epithelia. Modulation of ion fluxes in neurons and epithelia inhibits the perception of itch, pain, discomfort from the skin. By acting on these targets, preferred embodiment compositions are useful for skin and sensory disorders, and, in the case of secretory epithelia, to retard cellular proliferation. These compounds are formulated as a topical or oral preparation with prolonged duration of action.

### CROSS-REFERENCE TO RELATED APPLICATIONS

This application claims the benefits of U.S. Provisional Application No. 60/547,263, “N-Aryl_{S}-Carboxamide Compositions and Methods” filed on Feb. 23, 2004.

### BACKGROUND

1. Field

This disclosure generally relates to chemicals that affect sensory processes. These chemicals also activate receptors of the TRP (transient receptor potential) ion channel family, more particularly to the subgroup TRP-M8 and TRP-A1 receptors that are present in sensory nerves. More particularly, this disclosure provides peripheral sensory compositions that are useful in sensory refreshment and alleviation of skin irritation, itch and pain. These compositions have surprising pharmacokinetic properties that allow a prolonged duration of pharmacologically actions.

2. Description of Related Art

About two to three decades ago a group of scientists discovered novel compounds that have a physiological cooling action on the skin. These were described in U.S. Pat. Nos. 4,193,936 (Watson et al., Mar. 18, 1980), 4,248,859 (Rowsell et al, Feb. 3, 1981) and 4,318,900 (Rowsell, Mar. 9, 1982). More recently physiological receptors for cooling and cold sensations were discovered, one being an 1104-amino acid protein named TRP-M8, and another receptor called TRP-1 (also known as TRP-ANKM1).

There are currently two major classes of drugs that act peripherally to reduce perception of nociceptive signals in the central nervous system; nociceptive signals being stimuli that cause irritation, itch and pain. One class is the local anesthetics, such as procaine and lidocaine, which inhibit peripheral nerve conduction of nociceptive signals towards the central nervous system. Another group is agents like aspirin and ibuprofen that inhibit the synthesis of certain prostaglandins. These prostaglandins when released by tissues during injury or inflammation lower the threshold of firing of sensory nerve fibers that respond to noxious stimuli. By “peripheral” is meant that the target of the drug action is located outside the central nervous system, that is, outside of the brain and spinal cord. By “antinociceptive” is meant that the drug suppresses the psychical and physiological perception of noxious stimuli.

The sensory fibers that code for thermosensation, irritation, itch and pain are small-diameter sensory fibers called Aδ and unmyelinated C fibers. They are also sometimes called polymodal. When tissues are irritated, injured or inflamed, the C fibers are especially activated. When the compounds of this invention are used, cooling and cold sensations are activated presumably by Aδ afferents. The activation process may be mediated by TRP-M8 and TR-P-A1 receptors on the sensory nerves. The cooling and cold process has the net effect of reducing perception of C fiber activated nociceptive signals.

### SUMMARY

In one aspect of the present discovery, compounds are described here that have cooling and cold effects that are useful to counteract sensory irritation, itch and pain.

A particularly preferred set of embodiments are N-(substituted-aryl-alkyl)cycloalkyl-carboxamides. These particulary preferred embodiments have the desirable quality of long duration of action (more than 2 hours) when applied to the skin. Other advantages and aspects of the present disclosure will be understood by reading the following detailed description and the accompanying claims.

### BRIEF DESCRIPTION OF THE DRAWING

FIG. 1 graphically illustrates calcium entry into cells that were incubated with an embodiment of this invention as a function of time.

### DETAILED DESCRIPTION

Introduction.

Without being bound by theory, I believe that compounds in compositions of the present disclosure act on sensory processes in peripheral neurons to suppress perception of skin irritation, itch and pain. In normal skin, these compounds generate cooling, refreshing sensations. Embodiments are compounds with the structures shown in Formula 1 and examples in Formula 2. A novel feature of the structures described here is that R′ is C₁ to C₃ n-alkyl and not a hydrogen. The consequence of this feature is a more prolonged duration of topical action. Entities described by Watson et al. vide supra when applied directly to the skin in a single dose generally act for less than 1 hour. The chemicals described here when applied once topically have actions that can last for three to six hours or more.
R—CO—NH—R′—Y Formula 1
where

a) R is (1R,2S,5R)-2-Isopropyl-5-methyl-cyclohexyl,

b) R′ is C, to C₃ n-alkyl,

c) Y is an substituted aryl, or heterocyclyl,
where the aryl or heterocyclyl includes phenyl, 1-naphthyl, indenyl, azulenyl, heptalenyl, indacenyl, pyridinyl, dihydropyridinyl, pyridazinyl, piperazinyl, pyrmidinyl, pyrazinyl, indolyl, purinyl, indolizinyl, quinolinyl, isoquinolinyl, quinazolinyl, carbazolyl, pyrrolyl, thiazolyl, isothiazolyl, imidazolyl, benzothiophenyl, and phenathridinyl; and
where one to five of the substituent(s) on the aryl or heterocyclyl being one or more of halogen, or C₁ to C₈ alkyl, or alkenyl, or hydroxyl, or C₁ to C₈ alkoxy, or C₂ to C₁₀ alkylcarbonyloxy, or C₂ to C₁₀ carboxyalkyl or alkylcarboxyalkyl, or C₃ to C₁₀ alkylcarbonyloxyalkyl, or C₂ to C₈ acyl, or amino, C₁ to C₈ alkylamino, or C₂ to C₁₀ acylamino, or sulfonamido or C₁ to C₈ alkylsufonylamino, or N-arylsulfonamido or N-heterocyclylsulfonamido and where the aryl or heterocyclyl is selected from the group phenyl, benzyl, oxazoyl, thiazoyl, pyrimidinyl, pyridazinyl, 1,2,4-triazinyl, and where the aryl or heterocyclyl moiety is optionally substituted with a) up to three C₁ to C₃ alkyl groups, b) up to three C₁ to C₃ alkoxy groups, c) C₁ to C₈ aminoalkyl or diaminoalkyl, d) C₂ to C₁₀ alkylaminoalkyl, e) C₂ to C₁₀ acylaminoalkyl, f) carboxy, or g) C₂ to C₁₀ alkylcarboxy.

The carboxamide group is preferably in an equatorial position relative to the plane of the cycloalkyl ring. The aryl or heterocyclyl ring permits multiple radical insertions, which increases the versatility of the ligand for its receptor.

Compounds which are especially effective in the practice of this discovery are shown in Formula 2. 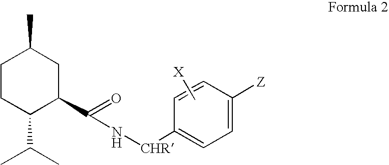
Formula 2. N-substituted-phenyl-alkyl-(1R,2S,5R)-2-isopropyl-5-methyl-cyclohexanecarboxamide (synonym: (1R,2S,5R)-2-Isopropyl-5-methyl-cyclohexanecarboxylic acid alkyl-substituted-phenylamide) where R′ is hydrogen or C₁ to C₂ n-allyl; X is hydroxyl, or C₁ to C₃ alkyl, or C₁ to C₃ hydroxyalkyl, or C₁ to C₃ alkoxy; and Z is C₁ to C₃ alkoxy, sulfadiazinyl, or carboxylic acid methyl, ethyl or propyl ester, Examples are: 4-Methoxy-N-benzyl-(1R,2S,5R-2-isopropyl-5-methyl-cyclohexanecarboxamide; 4-Hydroxy-3-methoxy-N-benzyl-(1R,2S,5R)-2-Isopropyl-5-methyl-cyclohexanecarboxamide; and 4-Methoxy-N-1-ethylphenyl-(1R,2S,5R)-2-isopropyl-5-methyl-cyclohexanecarboxamide (respective synonyms are: (1R,2S,5R)-2-Isopropyl-5-methyl-cyclohexanecarboxylic acid 4-methoxy-benzylamide; (1R,2S,5R)-2-Isopropyl-5-methyl-cyclohexanecarboxylic acid 4-hydroxy-3-methoxy-benzylamide; and (1R,2S,5R)-2-Isopropyl-5-methyl-cyclohexanecarboxylic acid [1-(4-methoxy-phenyl)ethyl]-amide)
Use of N-(Substituted-Arylalkyl)-Cycloalkyl Carboxamides

Hypothesis on Refreshment and Relief of Sensory Discomfort and Novelty.

I believe that the N-(substituted-arylalkyl)-cycloalkyl carboxamides (abbreviated here as N-aryls_{S}alkyl-cCarboxamide) and synonymous with cycloalkyl carboxylic acid-aryl-R′-substituted amide, where R′ is methyl, ethyl, n-propyl (and the corresponding terminology is benzylamide, phenyethylamide, phenyl-1-propylamide) act on specific receptors to stimulate ion channels of peripheral sensory neurons. For example, it is known that ion channel receptors, belonging the TRP (transient receptor potential) family of proteins, may mediate the effects of cooling on sensory neurons (TRP-M8, TRP-A1). When these ion channel neurons are activated, a subset of nerve fibers called Aδ cold fibers relay signals to the spinal cord and brain. These signals generate sensations of coolness and refreshment. If pathological conditions are present, these signals also suppress perception of signals for tissue irritation, for pruritus, and for acute and chronic pain.

Without being limited by theory an analogy of operation is as though there were three telephone lines in the tissues, each with a different dialing mechanism and cable conduction system. One is for touch and pressure that is fast conducting. One for coolness and cold that is somewhat slower (Aδ conducts at about 2 to 6 meters/sec). One for irritation, itch and pain that conducts slowly (<2 meters/sec, primarily C-fibers). It is known that acupuncture and scratching may relieve pain and itch via vibration and pressure, respectively, and that this process takes place at the level of the spinal cord (the so called “gate-control theory of pain”). In the analogy, one of two telephone lines interferes with the signaling of the other, but at the central exchange. Continuing the analogy of this disclosure, I propose the use of compounds of this discovery as the dialing mechanism for stimulating the telephone line responsible for signals of coolness and cold. Using this new telephone line, I anticipate either a refreshing and cool signal to be generated and, in the presence of inflammation and injury, an antinociceptive effect that has benefit.

The compounds of this discovery are active at μg to mg/mL (nano to microM) concentrations when applied to the topical surfaces of the body. By topical I mean that the application is onto surfaces of the body in contact with air, which includes the skin, the eye surface, the upper and lower respiratory tract, and the entrance and exit of the gastrointestinal tract, that is, the oral cavity and the anorectum. A second feature of these compounds is a longer duration of action, on the order of several hours, relatively to compounds described previously which are active for less than one hour. The longer duration of action was discovered by using a different bioassay: application of the test compounds to the skin of test subjects, and not by using taste thresholds for coolness or by using in vitro receptor assays.

Bioassays for Activity of N-Alkylaryl_{S}-Alkyl/Cycloalkyl Carboxamides

Tests for Activities of New Compounds.

Pain was defined by Sir Charles Sherrington as “the psychic adjunct of an imperative protective reflex”. Psychic events such as cooling, irritation, itch, and pain cannot be expressed by animals (animals cannot say “ouch” or that “it itches”) so the sensory effects of chemicals must be indirectly inferred. Receptor assays, based on cells transfected with the gene for TRP-M8 or TRP-A1, may be used as a model of sensory processes. Such data are shown in Table 1 under the heading “EC50 FM TRP-M8 receptor”. The EC50 is the median effective concentration for producing a half-maximal response. These data are precise: but, as noted below, these assays give no information on how long and for what quality of sensations will occur on human sensations. The best information on the pharmacological properties of these chemicals must therefore be derived from human experiment.

In humans, Rowsell et al. tested the properties of N-aryl_{S}-cCarboxamide by putting filter paper (1×1 cm), impregnated with a known amount of compound onto the dorsal surface of the tongue of the test subject. After 30 sec, the subject was required only to report presence or absence of a cooling effect. These data are reported as “Cool Threshold Tongue, ag” in Table 1 and refer to the threshold amount of the test substance that produces cooling sensations upon application onto the tongue of a panel of human volunteers. The full range of cycloalkyl structural variations and on substitutions on the phenyl and aryl rings, as it relates to cooling thresholds on the tongue is shown in Table 2.

I have found that the cooling and sensory properties of a given compound, including the novel N- aryls_{S}alkyl-cCarboxamide of this discovery, may be best obtained by suspending or dissolving a test substance in an ointment (usually Aquaphor® ointment which is 41% petrolatum, and the rest mineral oil, ceresin and lanolin alchol) and applying the emulsion (e.g. 0.2 to 0.5 cc) onto the skin surface. A reliable place for topical application is on the upper lip, above the vermillion border of the lips, on the philtrum, lateral to the philtrum until the nasolabial folds, and on the lower nostrils. This part of the face is known to be densely innervated with cold receptors, second only to the surface of eyeball. Tingling, cool and cold sensations may be experienced and rated for intensity. This test is quite accurate for predicting the cooling activity of candidate N-aryls_{S}alkyl-cCarboxamide drugs, but most importantly, it also measures the duration of drug action on the sensory receptors. The duration of action is not measured in a receptor assay and is difficult to quantify on the tongue because of the dynamic fluid conditions in the oral cavity and the presence of taste factors that affect thermosensation.

Structure-Activity Relationships.

As earlier noted, Rowsell et al. and Watson et al. (vide supra) described certain carboxamides that have cooling effects. The bioassay data for the effects of these compounds on the tongue of humans are shown in Table 2. Watson et al. discussed the structural features of molecules that produce menthol-like cooling effects which included N-alkyl- and N-aryl_{S}-cCarboxamides but N-aryls_{S}alkyl-cCarboxamide were not synthesized and tested. For cooling action, the active compounds required a branched chain hydrophobic carbon unit and a —CONH— or —COO— moiety for hydrogen bonding. These were the minimum structural features essential for bioactivity and constituted the pharmacophore unit. Simple ethyl or methyl N-substituents in, for example, p-menthane-carboxamides produced cooling on the tongue with thresholds at 0.2 and 1.1 μg, respectively. Watson et al. and Roswell et al. did not address the qualitative nature of the cooling and cold sensations elicited, that is, the intensity of the cold, or the effects of substituents on the duration of the cooling or cold effects.

Qualitative Aspects of Cool and Cold Intensity.

The temperature of the skin and its environs gives rise to sensations that are qualitatively distinct. Thus, the normal skin temperature is 93 F.° (33.9 C.°) and when water is applied to the skin it is called tepid at 80 to 93 F.° (26.7 to 33.9 C.°); cool between 65 to 80 F.° (18.3 and 26.7 C.°), cold at 55 to 65 F.° (12.8 to 18.3 C.°), and very cold below 55 F.° (12.8 C.°) [Bierman: Therapeutic Uses of Cold. J. Amer. Med. Assoc. vol. 157, 1189-1192, 1955]. the lowering of skin temperature is accompanied by behavioral and emotional responses. Thus, at room temperatures at or below 65 F.°, an individual frequently seeks to turn on the thermostat. At ambient skin temperatures at or below 55 F.°, the sensations are aversive and accompanied by affect; that is, the person considers these cold sensations to be unpleasant, seeks to escape the environment, and may become angry, hostile, or malaised if escape is not possible. The emotional response is also influenced by the circumstances and the site of exposure. For example, ice cream inside the mouth is not aversive on a short-term basis, but an ice cube placed on the skin for more than few minutes is unpleasant. On the other hand, breathing cool air between 65 to 80 F.° is refreshing and alerting, especially if there is inflammation in the nasal cavity or if the skin temperature is elevated above normal,

I have found the N-aryl_{S}-cCarboxamide, CPS-112 (also known as WS-12), with a 4-methoxy- substitution on the phenyl ring, is active at sub-micromolar concentrations on the TRP-M8 assay (see Table 1). This compound elicits strong cold sensations when applied at 1 to 2% to the philtrum and its environs. The sensations are more cold than cool, and have the sting and harshness of high concentrations of menthol. The duration of action of a 2% preparation peaks at 30 min and is dissipated at the end of one hour. By contrast, the 4-ethoxy or 4-n-propoxy substituted analogs were qualitatively more cooling than cold, and have longer durations of action, averaging about 2.5 hours. The coolness and longer duration of action were also present in the N-aryls_{S}alkyl-cCarboxamide analogs. The reasons for the qualitative differences may be pharmacokinetic, that is, access and residence of the agonist molecule at the receptor sites. Thus, the analogs with a larger N-phenylalkyl- moiety and a longer alkoxyphenyl ring subsituent, may have slower onset and offset of action at the nerve endings. There may also be receptor selectivity, as I have found that CPS-112 acts on both TRP-M8 and TRP-A1, whereas the sulfadiazinyl analog, CPS-125 acts only on TRP-M8 (U.S. application Ser. No. 11/025547: Inventor, E. T. Wei, Aryl-substituted derivatives of cycloalkyl and branched chain alkyl carboxamides and carboxylic acids useful as antinociceptive drugs for peripheral targets, filed Dec. 27, 2004). The sulfadiazinyl analog produces only coolness and not cold, and it has a prolonged duration of action, averaging more than 4 hours (see Table 1).

The results here also provide new insight in the choice of the substituent(s) to be inserted onto the phenyl ring. The bioactivity is influenced by the electron withdrawing or electron donating properties of the aryl substituent. For example, CPS-129, a compound with a strong electron withdrawing 4-nitro group, in inactive on the receptor, yet the 4-nitrophenyl- analog is active on the tongue at a threshold dose of 0.3 to 2.0 μg. Similarly, the 4-fluorophenyl analog is active at 0.5 μg on the tongue and had an EC50 of 1.3 μM on the TRP-M8 receptor, yet its effects when applied via the philtrum assay was only transient coolness. An analysis of the structure-activity relationships from the present studies indicate that electron donating substituents on the aryl ring preferentially enhance coolness and prolong duration of action. This insight into structure-activity relationships for skin cooling effects are reflected in the choice of the preferred entities in Formula 2.

For topical uses, either as a cosmetic or as a therapeutic, it is desirable to have more coolness than cold, more cold than intense cold, and to have a duration of action that permits a practical application. For example, in cosmetic use, if the added compound is used to reduce the irritation produced by retinoids or α, or ω- fatty acids, then the duration of action should be at least three hours. The sensation should be of coolness and not of cold. These qualitative aspects of sensation are met by CPS-116 and CPS-125, but not by CPS-112 which produces sensations of intense cold. If the ointment is to have therapeutic value in the pruritus caused by xerosis in the elderly, in atopic eczema, or in perianal inflammation, then the duration of action is preferably more than six hours. Again, the philtrum method of testing gives an answer for the duration of action and the quality of sensation. The improvement in drug action, provided by the N-aryls_{S}alkyl-cCarboxamide and the choice of ring substituents,, as revealed in the data presented here, are unexpected, surprising and have not been previously disclosed.

TABLE 1
Bioassay results on cooling compounds.

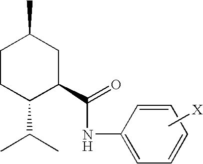

| | | | | |
|---|---|---|---|---|
| | | Cool | EC50 μM, | |
| | | Threshold | TRP-M8 | Philtrum, |
| Code Names | Structures | Tongue, μg | receptor | Duration , Hr |

X is:

| | | | | |
|---|---|---|---|---|
| CPS-128 | 4-ethoxy- | 0.1 | 0.5 | ˜1 |
| CPS-112 | 4-methoxy- | 0.1 | 0.6 | <1 |
| CPS-113 | 3-fluoro, 4-methoxy- | 0.3 | 1.3 | <1 |
| CPS-124 | 4-fluoro- | 0.5 | 1.3 | <1 |
| CPS-125 | 4-sulfadiazinyl- | 2.0 | 6 | 4+ |
| CPS-141 | 3-methoxy- | — | 6 | <1 |
| CPS-123 | 4-bromo- | >10 | 6 | NR |
| CPS-120 | 4-iodo- | >10 | 10 | NR |
| CPS-131 | 4-sulfadimethoxinyl- | >100 | 60 | <1 |
| CPS-127 | 4-trifluoromethoxy- | NR | NR | NR |
| CPS-129 | 3-trifluromethyl, 4-nitro- | 2.0 | NR | NR |
| CPS-132 | 4-sulfisoxazolyl- | NR | NR | <1 |
| CPS-126 | 4-sulfanilamidyl- | NR | NR | NR |

This invention:

| | | | | |
|---|---|---|---|---|
| CPS-116 | (1R,2S,5R)-2-Isopropyl-5-methyl- | 15 | 8 | 3+ |
| | cyclohexanecarboxylic acid 4- | | | |
| | hydroxy-3-methoxy-benzylamide | | | |

Other Compounds

| | | |
|---|---|---|
| menthol | 25 | <1 |
| icilin | 0.8 | 5+ |

NR = no response

TABLE 2
Sensory thresholds for various N-arylₛ-cCarboxamide.

| | Bioactivity |
|---|---|
| | Tonguc*, |
| CHEMICAL | μg |
| N-(3′-hydroxy-4′-methylphenyl)-2-isopropyl-5-methylcyclohexanecarboxamide | 0.1 |
| N-(4′-methoxyphenyl)-2-isopropyl-5-methylcyclohexanecarboxamide | 0.1 |
| N-(2′,4′-dimethylphenyl)-2-isopropyl-5-methylcyclohexanecarboxamide | 0.1 |
| N-(4′-methoxy-2′-methylphenyl)-1-isopropylcycloheptanecarboxamide | 0.2 |
| N-(4′-methylphenyl)-2-isopropyl-5-methylcyclohexanecarboxamide | 0.3 |
| N-(4′-nitrophenyl)-2-isopropyl-5-methylcyclohexanecarboxamide | 0.3 |
| N-(3′,4′-dimethoxyphenyl)-2-isopropyl-5-methylcyclohexanecarboxamide | 0.3 |
| N-(2′-hydroxyphenyl)-2-isopropyl-5-methylcyclohexanecarboxamide | 0.5 |
| N-(4′-fluorophenyl)-2-isopropyl-5-methylcyclohexanecarboxamide | 0.5 |
| N-(4′-methoxyphenyl)-2-isopropyl-2,3-dimethylbutyramide | 0.5 |
| N-(2′-methoxyphenyl)-2-isopropyl-5-methylcyclohexanecarboxamide | 0.5 |
| N-(3′-hydroxy-4′-methylphenyl)-1-isopropylcycloheptanecarboxamide | 1 |
| N-(2′-methyl-4′-methoxyphenyl)-3-n-butylbicyclo[2.2.1]hept-5-ene-2-carboxamide | 1 |
| N-(2′,4′-dimethoxyphenyl)-3-isobutylbicyclo[2.2.1]hept-5-ene-2-carboxamide | 1 |
| N-(4′-hydroxyphenyl)-2-isopropyl-5-methylcyclohexanecarboxamide | 1 |
| N-(3′-pyridyl)-2-isopropyl-5-methylcyclohexanecarboxamide | 1 |
| N-(2′,4′-dimethylphenyl)-2-isopropyl-2,3-dimethylbutyramide | 1 |
| N-(2′-methyl-4′-methoxyphenyl)-3-isobutylbicyclo[2.2.1]hept-5-ene-2- | 1 |
| carboxamide | |
| N-(4′-acetylphenyl)-2-isopropyl-5-methylcyclohexanecarboxamide | 2 |
| N-(4′-methoxyphenyl)-2-isopropyl-2,4-dimethylpentanamide | 2 |
| N-(4′-methylphenyl)-2-isopropyl-5-methylcyclohexanecarboxamide | 3 |
| N-(3′,4′-dimethylphenyl)-2-isopropyl-2,3-dimethylbutyramide | 3 |
| N-(4′-methoxyphenyl)-3-isobutylbicyclo[2.2.1]hept-5-ene-2-carboxamide | 4 |
| N-(3′-hydroxy-4′-methoxyphenyl)-3-n-butylbicyclo[2.2.1]hept-5-ene-2- | 5 |
| carboxamide | |
| N-(2′,4′-dihydroxypyrimidin-5′-yl)-2-isopropyl-5-methylcyclohexanecarboxamide | 5 |
| N-(3′,4′-dimethoxyphenyl)-1-isopropylcycloheptanecarboxamide | 5 |
| N-(4′-ethoxycarbonylphenyl)-2-isopropyl-5-methylcyclohexanecarboxamide | 5 |
| N-(2′,5′-dimethylphenyl)-2-isopropyl-2,4-dimethylpentanamide | 5 |
| N-(4′-methoxyphenyl)-1-ethyl-2-methylcycloheptanecarboxamide | 6 |
| N-(1′-naphthyl)-2-isopropyl-5-methylcyclohexanecarboxamide | 6 |
| N-(3′-hydroxy-4′-methoxyphenyl)-3-isobutylbicyclo[2.2.1]hept-5-ene-2- | 8 |
| carboxamide | |
| N-(4′-methoxyphenyl)-1,7,7-trimethylbicyclo[2.2.1]heptane-2-carboxamide | 8 |
| N-(4′-chlorophenyl)-2-isopropyl-5-methylcyclohexanecarboxamide | 8 |
| N-(3′-hydroxyphenyl)-1-isopropylcycloheptanecarboxamide | 10 |
| N-(2′,4′-dimethylphenyl)-1-isopropylcycloheptanecarboxamide | 15 |
| N-(3′,4′-dimethylphenyl)-1-isopropylcycloheptanecarboxamide | 15 |
| N-(4′-methoxyphenyl)-3-isopropylbicyclo[2.2.1]heptane-2-carboxamide | 15 |
| N-(4′-methoxyphenyl)-3,3-dimethylbicyclo[2.2.1]heptane-2-carboxamide | 20 |
| N-(2′,5′-dimethylphenyl)-3,3-dimethylbicyclo[2.2.1]heptane-2-carboxamide | 20 |
| N-phenyl-2-isopropyl-5-methylcyclohexanecarboxamide | 20 |
| N-phenylmethyl-2-isopropyl-5-methylcyclohexanecarboxamide | 20 |
| *Filter paper (1 × 1 cm) was impregnated with a known amount of compound and placed on the tongue of the test subject. After 30 sec, the subject was required only to report presence or absence of a cooling effect. Bioactivity (μg) refers to | |
| # the threshold amount of the test substance that produces cooling sensations upon application onto the tongue of a panel of human volunteers | |

Actions on Secretory Epithelium.

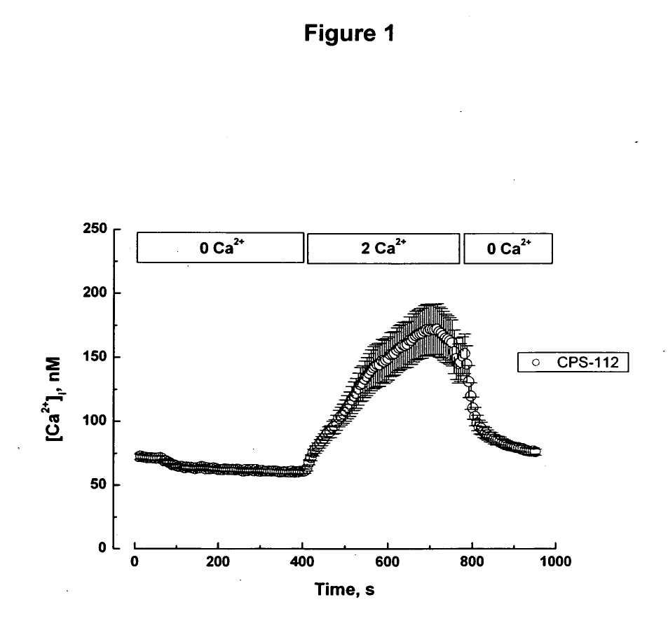

Certain cancer cell lines, such as (LNCaP, for lymph node-derived prostate cancer cells) constitutively express functional binding sites on their membrane surfaces. The unknown receptors react to the N-aryl_{S}-cCarboxamides and to the N-aryls_{S}alkyl-cCarboxamide of this invention. In a LNCaP cell line which does not express the TRP-M8 gene, the EC50 for N-(4′-methoxyphenyl)-2-isopropyl-5-methyl-cyclohexanecarboxamide (CPS-112) on stimulated calcium entry into cells was found to be 2 nanoM. The results are illustrated in FIG. 1. A similar finding was reported by Reynolds and Polakis, Therapeutic aspects of trp-p8 active compounds and therapeutic methods. Jan. 13, 2005, WO2005, 002582 A2.

With reference to FIG. 1, lymph-node derived human prostate cancer cells (LNCaP) were incubated with 2 nanoM of N-(4′-methoxyphenyl)-2-isopropyl-5-methyl-cyclohexanecarboxamide (CPS-112). These cells were then incubated with a calcium fluorescent marker and in the presence of CPS-112 a robust calcium entry response was obtained when calcium ions were added. When the calcium was washed away from the medium, the entry of the cation declined. Vehicle treated cells did not show such effects.

Calcium stores and fluxes across membranes are important determinants of cell growth and death. Certain TRP receptors, such as TRP-M8 (also known as trp-p8) and TRP-V6, have been identified in secretory epithelial cells such as the liver, pancreas, and prostate. It is recognized in a number of publications that targeting of these receptors may offer a new approach to the treatment of cancer (Peng et al. Epithelial Ca2+ entry channels: transcellular Ca²⁺ transport and beyond. J. Physiol. 551, 729-740, 2003; Fixemer et al. Expression of the Ca²⁺-selective cation channel TRPV6 in human prostate cancer: a novel prognostic marker for tumor progression. Oncogene 22: 7858-7861, 2003; Vanden Abeele et al. Store-operated Ca²⁺ channels in prostate cancer epithelial cells: function, regulation, and role in carcinogenesis. Cell Calcium 33: 357-373, 2003, and Laus et al. (Prostate tumor nucleotide compositions and methods of detection thereof U.S. Pat. No. 6,194,152 B1, Feb. 27, 2001). The activity of the N-Aryls_{S}alkyl-cCarboxamide of this discovery on LNCaP cells (data not shown) suggests that these compounds may have also be useful in treating malignancy, as well as providing therapeutic opportunities in disorders of the epithelium, osteoporosis, calcium deficiency states, and diabetes mellitus, although the precise TRP receptor targets for these diseases have not yet been identified. Hence, the oral administration of these compounds for therapeutic use is also contemplated.

Delivery to Target and Therapeutic Applications of N-benzyl_{S}-cCarboxamide Embodiments

In practicing this discovery the N-aryls_{S}alkyl-cCarboxamide compounds, applied topically to inflamed skin and mucous membranes, will typically relieve itch, irritation and pain. By “topical” is meant application onto surfaces of the body in contact with air, which includes the skin, the eye surface, the upper and lower respiratory tract, and the entrance and exit of the gastrointestinal tract, that is, the oral cavity and the anorectum Suitable topical formulations, for example, include compositions such as powders, pastes, lotions, liniments, creams and ointments, and cosmetic preparations.

In formulating topical compositions to practice this discovery, the N-aryls_{S}alkyl-cCarboxamide may be incorporated into a vehicle that by itself may be inert or may contain other active ingredients (e.g. a fragrance or a glucocorticosteroid). A wide variety of vehicles will be suitable, depending upon the particular product involved, such vehicles including solids, liquids, emulsions, foams and gels. Typical vehicles include oils and fats such as hydrocarbon oils, fatty acid esters, long chain alcohols and silicone oils; finely divided solids such as starch or talc; low-boiling hydrocarbons; gums and natural or synthetic resins. For applications to the ocular surface or to the upper or lower respiratory tract, the compound may be packaged in unit dose dispensers.

Therapeutic indications for which a topical formulation may be beneficial include irritation, itch and pain from various forms of dermatitis (atopic, contact and irritant); pain from burned, traumatized or irritated skin, from procedures related to wound debridement; itch and discomfort from skin infections, insect bites, sunburn, actinic keratoses, basal cell carcinoma, pruritus due to xerosis; cheilitis or itching of the lips from cold sores; pruritus ani, hemorrhoidal discomfort, pain from anal fissures, pain or itch from anal fistulas, pain from hemorrhoidectomy, perineal inflammation, anogenital skin inflammation and discomfort due to various local causes such as incontinence, diaper rashes, perineal inflammation; vulval pruritus and pain (e.g. from candidiasis or idiopathic, such as vulva vestibulitis and vulvodynia), dyspaurenia, anogenital infections, including warts and sexually transmitted diseases, viral infections of the skin (especially in immunocompromised patients); nostril and nasal or upper airway discomfort from breathing obstruction, e.g. rhinitis, asthma, bronchitis, emphysema and chronic obstructive pulmonary diseases, sleep apnea and snoring; conjunctivitis, pain from corneal abrasions, and pain from eye surgery.

If the target is to be reached via the bloodstream, an oral formulation is designed to be optimally absorbed from the gastrointestinal tract and to achieve steady blood or plasma levels. Here again, a simple gelatin capsule or an enteric coated pill or capsule, designed for optimum dissolution at a given pH, is a familiar formulation to practitioners skilled in the art. Extensively used chemicals for enteric coating are cellulose acetate phthalate, methacrylic acid ester copolymers with acidic ionizable groups, and polyvinyl acetate phthalate. Standard coating ingredients are widely sold under the trademark of Eudragit® (Degussa Chemicals, Inc.). Dosage forms coated with methacrylic acid polymers dissolve in the ileum at about pH 6.8, and in the terminal ileum and caecum at about pH 7.2. In general coating thicknesses of about 25 to 200 microns, and especially 75 to 150 microns, are preferred using about 3 to 25 mg, preferably 8 to 15 mg of acidic coating material per square centimer of tablet or capsule surface. The precise coating thickness will however depend upon the solubility characteristics of the material used and the site to be treated.

The N-aryls_{S}alkyl-cCarboxamide compositions described here have the desirable properties of non-irritancy, safety and long duration of action. Uses of an oral formulation containing the inventive embodiments would include conditions such as heat exhaustion and fatigue, nasal and eye irritation, obstructed breathing disorders, lower urinary tract disorders, heartburn, irritable bowel disease or the irritable bowel syndrome, generalized pruritus, and systemic pain.

### EXAMPLE 1

Synthesis of N-aryls_{S}alkyl-cCarboxamide.

Synthesis of the carboxamides is usually achieved by reaction of the free amine with the appropriate acid chloride in the presence of a suitable acceptor for hydrogen chloride, which could be an excess of the free amine or another base, e.g. triethylamine. The reaction is carried out in a suitable organic solvent, but, depending on the reactivity of the acid chloride, may also be carried out in a mixed aqueous/organic solvent system, in which case a convenient base is sodium bicarbonate. The synthesis commences with the reaction of a carbonyl chloride with the appropriate arylalkylamine derivative. The acid chloride, or carbonyl chloride, may be obtained by many methods known to the art. As an example, 2-isopropyl-5-methylcyclohexane carbonyl chloride is prepared from L-menthol, via the following route: firstly, reaction with zinc chloride in hydrochloric acid to prepare 2-isopropyl-5-methylcyclohexyl chloride; next preparation of the Grignard reagent and carbonation to yield 2-isopropyl-5-methylcyclohexane carboxylic acid; and finally reaction with thionyl chloride to yield the carbonyl chloride.

Many substituted arylalkylamines may be obtained from commercial sources such Sigma-Aldrich Corp., St. Louis, Mos. For example, 2-ethoxybenzylamine, 4-methoxybenzylamine, 4-methoxyphenylethylamine, and 3-methoxyphenylpropylamine are listed in the 2003-2004 Aldrich Catalog. The acid chloride is reacted with the appropriate arylalkylamine to form the N-aryl_{S}alkyl-cCarboxamide.

Synthesis of 4-Hydroxy-3-methoxy-N-benzyl-(1R,2S,5R)-2-Isopropyl-5-methyl-cyclohexanecarboxamide (synonym:(1R,2S,5R)-2-Isopropyl-5-methyl-cyclohexanecarboxylic acid 4-hydroxy-3-methoxy-benzylamide). 1 mmol (0.19 g) of 4-hydoxy-3-methoxy-benzylamine HCl (Sigma Aldrich, Co.) was dissolved in 4.5 ml of ether in a 25 ml flask, and two equivalents (0.3 ml) of triethylamine was added and stirred, followed by 0.20 g (0.14 ml) of p-menthoyl chloride. The reaction vessel was first kept in ice for about 1 hr then stirred overnight at room temperature. The resulting product was extracted with ˜20 ml of ethylacetate and the organic layer washed 3× with ˜15 ml water. The aqueous layer was further extracted with ethylacetate, and the solvent dried with sodium sulfate, and the solids filtered out. The combined ethylacetate was evaporated and 0.326 g product was obtained. The purity of the product was ascertained by thin-layer chromatography (5:1, hexane:ethylacetate) and the structure confirmed by nuclear magnetic resonance. This compound was assigned the code of CPS-116.

The N-aryl_{S}alkyl-cCarboxamides of the present invention are mostly white crystalline solids at room temperatures. For bioassay of CPS-116 on the skin, 100 mg of the above product was stirred and dissolved in 5 g of warm liquid Aquaphor® ointment to a yield a 2% ointment. After cooling, the solid ointment was applied to the philtrum, and lateral to the philtrum, of test subjects with a glass rod and the onset and duration of cooling sensations noted. At this concentration (2%), CPS-116 produced cooling sensations that lasted for an average of 3+ hours in 5 trials. The cooling sensations were pleasant and were not accompanied by stinging or tingling sensations.

### EXAMPLE 2

The methods for the TRP-M8 and TRP-A1 receptor studies are described in Bevan et al. (Vanilloid receptor-related nucleic acids and polypeptides. U.S. Ser. No. 2003/0157633 A1, Aug. 21, 2003), Julius et al. (Methods of modulating cold sensory perception. U.S. Ser. No. 2003/0219834 A1, Nov. 27, 2003) and Behrendt et al. Characterization of the mouse cold-menthol receptor TRPM8 and vanilloid receptor type-1 VR1 using a fluorometric imaging plate reader (FLIPR) assay. Brit. J. Pharmacol. 2004 February;141(4):737-45. The data here were collected by Dr. Afrodite Lourbakos of Unilever Research and Development, Vlaardingen, the Netherlands, using similar transfection methods and a FLIPR assay system.

Human embryonic kidney cells were permanently transfected with the gene for the human TRP-M8 receptor. These cells were then incubated with a calcium fluorescence indicator (Fura-2) and incubated at either 29 or 37° C. These cells were then distributed into a 96-well fluorescence-plate image reader with automated drug dilution and computerized software for dose-response analysis. Calcium ion influx into cells after stimulation with compounds was quantified in fluorescence units. Compounds were dissolved in DMSO by ultrasonication to a 0.1 M solution. 5 μl of this stock was added to 5 mg of cyclodextrin and 5 ml of 140 Na-tyrode, to achieve various final test concentrations. Icilin and menthol, standard sensory nerve agonists with antinociceptive properties, were used as positive controls and gave median effective concentration activities of 0.8 and 25 μM (EC50) activities. The EC50 of CPS-116 in this test system was 8 μM. Further analysis of the dose-response relationship showed the ΔFmax (the maximum fluorescence increase induced by a compound at the maximum concentration tested) for CPS-116 of 14,000 units which was similar to that seen with icilin and menthol, confirming full activation of the receptor.

### EXAMPLE 3

A 36-year old with the common cold had severe rhinitis for a period of 4 days. Vigorous blowing of the nose resulted in a reddened, chapped, and painful area on the border of the nostrils, the philtrum, the area immediately lateral to the philtrum, and above the vermillion border of the lips. Application of a 2% CPS-116 ointment produced cooling sensations within 5 minutes and produced relief from irritation and pain for about 5 hours. This beneficial effect was repeated for 6 times in the same individual and was also observed in three other individuals with the common cold.

It is to be understood that while the disclosure has been described above in conjunction with preferred specific embodiments, the description and examples are intended to illustrate and not to limit.

1. A composition comprising a compound having the structure shown by Formula 1
R—CO—NH—R′—Y Formula 1
where
R is (1R,2S,5R)-2-Isopropyl-5-methyl-cyclohexyl,
R′ is C₁ to C₃ n-alkyl,
Y is an substituted aryl, or heterocyclyl,
where the aryl or heterocyclyl includes phenyl, 1-naphthyl, indenyl, azulenyl, heptalenyl, indacenyl, pyridinyl, dihydropyridinyl, pyridazinyl, piperazinyl, pyrmidinyl, pyrazinyl, indolyl, purinyl, indolizinyl, quinolinyl, isoquinolinyl, quinazolinyl, carbazolyl, pyrrolyl, thiazolyl, isothiazolyl, imidazolyl, benzothiophenyl, and phenathridinyl; and
where one to five of the substituent(s) on the aryl or heterocyclyl being one or more of halogen, or C₁ to C₈ alkyl, or alkenyl, or hydroxyl, or C, to C₈ alkoxy, or C₂ to C₁₀ alkylcarbonyloxy, or C₂ to C₁₀ carboxyalkyl or alkylcarboxyalkyl, or C₃ to C₁₀ alkylcarbonyloxyalkyl, or C₂ to C₈ acyl, or amino, C₁ to C₈ alkylamino, or C₂ to C₁₀ acylamino, or sulfonamido or C₁ to C₈ alkylsufonylamino, or N-arylsulfonamido or N-heterocyclylsulfonamido and where the aryl or heterocyclyl is selected from the group phenyl, benzyl, oxazoyl, thiazoyl, pyrimidinyl, pyridazinyl, 1,2,4-triazinyl, and where the aryl or heterocyclyl moiety is optionally substituted with a) up to three C₁ to C₃ alkyl groups, b) up to three C₁ to C₃ alkoxy groups, c) C₁ to C₈ aminoalkyl or diaminoalkyl, d) C₂ to C₁₀ alkylaminoalkyl, e) C₂ to C₁₀ acylaminoalkyl, f) carboxy, or g) C₂ to C₁₀ alkylcarboxy; and,
a vehicle for the compound suitable for topical application of the composition as an ointment.

2. The composition of claim 1 wherein R is (1R,2S,5R)-2-Isopropyl-5-methyl-cyclohexyl; R′ is C₁ to C₃ n-alkyl; and Y is a phenyl ring singly or multi-substituted with C₁ to C₃ alkoxy; or sulfadiazinyl; or carboxylic acid methyl, ethyl, or propyl ester; or hydroxyl; or C₁ to C₃ alkyl; or C₁ to C₃ hydroxyalkyl.

3. The composition of claim 1 wherein the compound is (1R,2S,5R)-2-Isopropyl-5-methyl-cyclohexanecarboxylic acid 4-sulfadiazinyl-benzylamide.

4. The composition of claim 1 wherein the compound is (1R,2S,5R)-2-Isopropyl-5-methyl-cyclohexanecarboxylic acid 4-carboxylic acid ethylester-benzylamide.

5. The composition of claim 1 wherein the compound is (1R,2S,5R)-2-Isopropyl-5-methyl-cyclohexanecarboxylic acid 4-hydroxy-3-methoxy-benzylamide.

6. A cosmetic method comprising topically administering the composition of claim 1.

7. The method as in claim 6 wherein from about 0.2 to about 5% by weight of the compound is topically applied.

8. A therapeutic method comprising orally administering the composition of claim 1.

9. The method as in claim 8 wherein the orally administered amount provides about 0.01 to 2 grams of the compound per dose.
